(19) United States
(12) Patent Application Publication
(10) Pub. No.: US 2025/0113982 A1
(43) Pub. Date: Apr. 10, 2025

(54) MEDICAL SYSTEMS, DEVICES, AND METHODS FOR SEPARATING LAYERS OF TISSUE

(71) Applicant: Boston Scientific Scimed, Inc., Maple Grove, MN (US)

(72) Inventor: Austin Grant JOHNSON, Worcester, MA (US)

(73) Assignee: Boston Scientific Scimed, Inc., Maple Grove, MN (US)

(21) Appl. No.: 18/907,951

(22) Filed: Oct. 7, 2024

Related U.S. Application Data
(60) Provisional application No. 63/589,096, filed on Oct. 10, 2023.

(51) Int. Cl. A61B 1/005; A61B 1/00

(52) U.S. Cl. CPC A61B 1/0057; A61B 1/00137

(57) ABSTRACT

A medical device including a handle with a first arm operator and a second arm operator, and a cap. The cap includes a body having a central lumen about a central longitudinal axis, a first arm hingeably coupled to the body and operably coupled to the first arm operator by a first wire, and a second arm hingeably coupled to the body and operably coupled to the second arm operator by a second wire. The first arm operator is moveable in the handle to move the first arm between a closed position with the first arm generally parallel to the central longitudinal axis and an open position with the first arm generally perpendicular to the central longitudinal axis. The second arm operator is moveable in the handle to move the second arm between a closed position and an open position.

### CROSS-REFERENCE TO RELATED APPLICATIONS

This application claims the benefit of priority to U.S. Provisional Application No. 63/589,096, filed on Oct. 10, 2023, which is incorporated by reference herein in its entirety.

### TECHNICAL FIELD

The disclosure relates generally to medical systems, devices, and associated methods for separating layers of tissue. More specifically, aspects of the disclosure pertain to devices or assemblies including components that are positioned at a distal tip of a medical insertion device, such as an endoscope.

### BACKGROUND

Medical endoscopy continues to advance, with endoscopic procedures becoming pivotal in diagnosing and treating a wide array of conditions. Third space endoscopy (TSE) offers a less-invasive means to explore deeper tissue layers without compromising the integrity of superficial structures. The existing tunneling process in TSE involves the introduction of a lifting agent to separate tissue layers and also includes a cutting knife to enable access.

TSE requires tunneling through a submucosa to a target site between the submucosa and the muscularis. This tunneling can be performed by a blunt object at a distal end of a shaft of an endoscope. The blunt object uses a mechanical force with a static object (e.g., an end cap) to separate tissue layers, thus tunneling through the submucosa to the target site. The static cap at the end of the endoscope shaft can introduce problems for users trying to reach a target site. For example, blunt tissue dissection with a static cap relies solely on the movement of the distal end of the shaft of the endoscope to separate tissue layers, which may limit the displacement or separation of tissue layers.

The systems, devices, and methods described herein may help solve one or more problems of the current art by providing endoscope caps with dynamic capabilities. The scope of the current disclosure, however, is defined by the attached claims, and not by the ability to solve any specific problem.

### SUMMARY

Each of the aspects disclosed herein may include one or more of the features described in connection with any of the other disclosed aspects.

In some aspects, a medical device includes a handle including a first arm operator; and a second arm operator; and a cap. The cap may include a body having a central lumen about a central longitudinal axis; a first arm hingeably coupled to the body and operably coupled to the first arm operator by a first wire; and a second arm hingeably coupled to the body and operably coupled to the second arm operator by a second wire. The first arm operator may be moveable in the handle to move the first arm between a closed position with the first arm generally parallel to the central longitudinal axis and an open position with the first arm generally perpendicular to the central longitudinal axis. The second arm operator may be moveable in the handle to move the second arm between a closed position with the second arm generally parallel to the central longitudinal axis and an open position with the second arm generally perpendicular to the central longitudinal axis.

In another aspect, the first arm and the second arm may be semi-circular arms. The first wire may be connected to the first semi-circular arm at an apex of the first semi-circular arm, and the second wire may be connected to the second semi-circular arm at an apex of the second semi-circular arm.

In another aspect, the first arm and the second arm may include metal or a hard plastic material. The first arm and the second arm may be biased toward an open position.

In another aspect, the first arm may be biased toward an open position by a first spring in the handle that biases the first arm operator, and the second arm may be biased toward an open position by a second spring in the handle that biases the second arm operator.

In another aspect, the cap may include a proximal portion configured to overlap with a distal portion of an insertion device. A distal portion may be configured to extend distally of the distal portion of the insertion device.

In another aspect, the insertion device may be an endoscope, and the cap may include an endoscope adaptor, and an endoscope cap for capping a shaft of the endoscope. The endoscope adaptor may be made of an elastic material, and the endoscope cap is made of a plastic material.

In another aspect, the endoscope adaptor is made of silicone, and the endoscope cap may be made of a clear material including ABS plastic, polycarbonate, or a similar plastic.

In another aspect, the first wire may be surrounded by a first tube, the second wire may be surrounded by a second tube, and proximal ends of the first tube and the second tube may be connected to the handle. The first tube and the second tube may each be made from a plastic extrusion.

In another aspect, a diaphragm that is connected between the first arm and the second arm and an endoscope cap may be moveable with the first arm and the second arm to form a barrier between the central lumen of the cap and an external environment with the first arm and the second arm in the closed position.

In another aspect, the cap may include one or more vent holes through the body of the cap.

In yet another aspect, a cap for an insertion device may include a body having a central lumen about a central longitudinal axis; a first arm hingeably coupled to the body and operably coupled to a first wire; and a second arm hingeably coupled to the body and operably coupled to a second wire. The first arm may be moveable between an open position with the first arm generally perpendicular to the central longitudinal axis and a closed position with the first arm generally parallel to the central longitudinal axis, and the second arm may be moveable between an open position with the second arm generally perpendicular to the central longitudinal axis and a closed position with the second arm generally parallel to the central longitudinal axis.

In yet another aspect, the first arm may be operably coupled to a first arm operator in a handle by a first actuation element; the second arm may be operably coupled to a second arm operator in the handle by a second actuation element; the first arm operator may be moveable in the handle to move the first arm; and the second arm operator may be moveable in the handle to move the second arm.

In yet another aspect, a diaphragm may be connected between the first arm and the second arm and an endoscope cap and the diaphragm may be moveable with the first arm and the second arm to form a barrier between the central lumen of the cap and an external environment with the first arm and the second arm in the closed position.

In still another aspect, a method of reaching a target treatment site using an endoscope may include navigating an endoscope having an endoscope cap to a target insertion site at an external surface of a first tissue layer; puncturing the first tissue layer at the target insertion site; injecting a lifting agent between the first tissue layer and a third tissue layer through the puncture; tunneling through the first tissue layer and the second tissue layer between the first tissue layer and the third tissue layer to a target treatment site with the endoscope. The tunneling may include tunneling using the endoscope cap including a body having a central lumen about a central longitudinal axis, a first arm hingeably coupled to the body and operably coupled to the first arm operator by a first wire, and a second arm hingeably coupled to the body and operably coupled to the second arm operator by a second wire.

In still another aspect, transitioning the first arm and the second arm from a closed configuration to an open configuration may include an operator urging the first arm operator and the second arm operator proximally on a handle to (1) move the first arm with the first arm operator and (2) move the second arm with the second arm operator to tunnel between the first tissue layer and the third tissue layer.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIGS. 1A-1B depict an exemplary medical device, according to one or more embodiments shown and described herein.

FIGS. 2A-2B depict an exemplary medical device inside a body lumen of a subject.

FIG. 3 depicts a handle assembly for an exemplary medical device such as that depicted in FIGS. 1A-1B.

FIGS. 4A-4C depict an exemplary cap assembly including a cap and an adaptor for an exemplary medical device, such as the exemplary medical device of FIGS. 1A-1B.

FIGS. 5A-5B depict another embodiment of an exemplary cap assembly including a cap and an adaptor for an exemplary medical device, such as the exemplary medical device of FIGS. 1A-1B.

FIG. 6 depicts one or more aspects of an exemplary method of using a medical device, such as the exemplary medical device of FIGS. 1A-1B.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values+/−10% of a stated value.

A medical device, such as an endoscope may be inserted into a body lumen (e.g., an esophagus) of a subject in order to perform a medical procedure. The distal tip of the medical device may include electronic assemblies including various components, such as lighting elements (e.g., light emitting diodes (LEDs), optical fibers or other light guides, or a combination thereof), imaging elements (e.g., cameras, other components having imagers, or other optical elements (e.g., lenses)), associated electronic elements (e.g., capacitors, diodes, resistors, and the like).

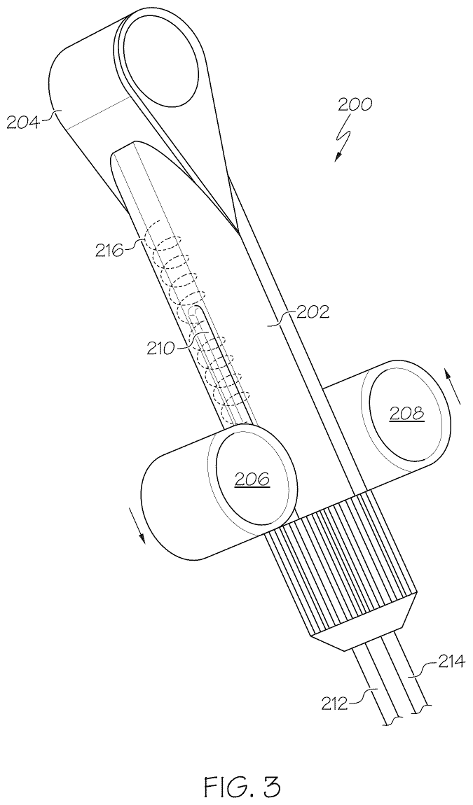

It may be desirable to cap the distal end of the medical device with one or more functional cap assemblies in order to assist in performing one or more medical procedures. One exemplary cap assembly may include an adaptor and a cap, with the cap having a pair of moveable arms. The moveable arms may be moveable using one or more wires. The one or more wires may be operatively coupled to the moveable arms to move the arms from a position generally perpendicular to a longitudinal axis of the medical device to a position generally parallel to the longitudinal axis of the medical device. The wires may operatively couple the moveable arms to a handle assembly with one or more moveable rings (e.g., the rings 206, 208 shown in, for example, FIG. 3). Movement of the rings can impart or otherwise affect movement of the moveable arms of the cap, thus giving an operator control over the moveable arms for use during a medical procedure.

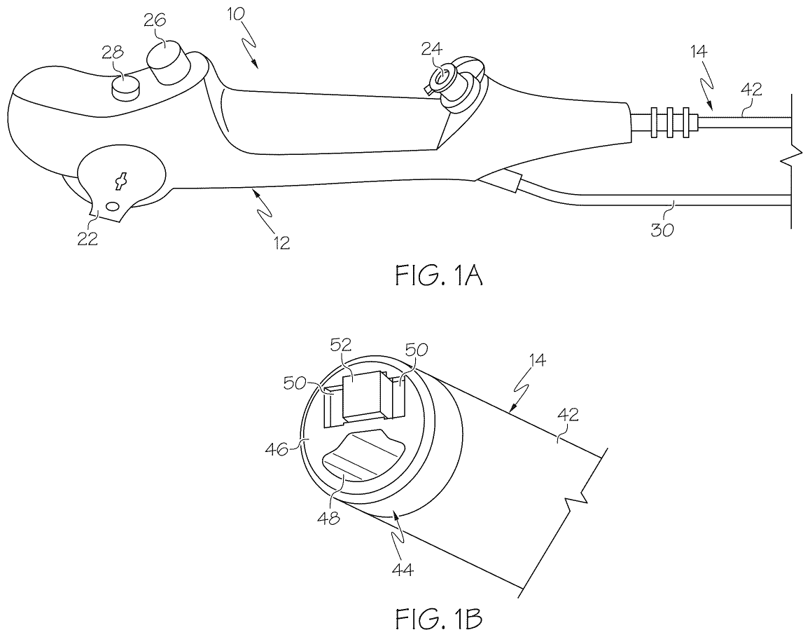

FIGS. 1A and 1B depict aspects of an exemplary medical device 10. FIG. 1A depicts a proximal portion of medical device 10. FIG. 1B depicts a distal tip 44 of medical device 10. Medical device 10 may include a handle portion 12 for gripping and operation by an operator, and an insertion portion 14 for at least partial insertion into a body (e.g., a body lumen) of a subject. As shown in FIGS. 1A and 1B, medical device 10 may include an endoscope and one or more other aspects of an endoscope system. As discussed in detail below, the distal tip 44 of the medical device 10 may include a cap assembly that may help the distal tip 44 advance between and/or separate layers of tissue. Although the disclosure may refer at different points to an endoscope, it will be appreciated that, unless otherwise specified, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the elements and assemblies described herein.

Handle portion 12 may include a lever 22, for example, on a proximal portion of handle portion 12. Lever 22 may help to facilitate articulation/steering of insertion portion 14, including distal tip 44. Although lever 22 is depicted in FIG. 1A, it will be appreciated that any suitable actuator(s) may be used in addition to or in place of lever 22, such as one or more knobs, buttons, sliders, or joysticks. A port 24 of handle portion 12 (e.g., on a proximal portion of handle portion 12) may provide access to a lumen or working channel of medical device 10. An operator may insert an instrument or other device into port 24 and may extend the instrument or other device distally through the working channel. The working channel may extend longitudinally through a length of insertion portion 14. Handle portion 12 may also include a suction valve 26, for example, on a proximal portion of handle portion 12 and on an opposing side from lever 22. An operator may connect suction valve 26 to a source of suction, and may operate suction valve 26 to generate suction through insertion portion 14 (e.g., through the working channel). Handle portion 12 may additionally or alternatively include other types of valves, such as air and/or water valves, or valves that perform a combination of functions.

An image capture button 28 of handle portion 12 may enable an operator to capture a still image from an imaging device 52 (shown in FIG. 1B and described in further detail below) during a procedure. Image capture button 28 may be positioned on a proximal portion of handle portion 12, for example, adjacent suction valve 26. Additionally or alternatively, image capture button 28 may enable an operator to capture video or to perform other functions to control medical device 10. An umbilicus 30 may extend from handle portion 12 (e.g., from a distal portion of handle portion 12) and may carry wires, cables, and/or conduits for providing, for example, power, signals, or fluids to and/or from handle portion 12. For example, umbilicus 30 may connect handle portion 12 to one or more user interfaces, monitors, displays, etc.

Insertion portion 14 may include a shaft 42 extending distally from handle portion 12. Shaft 42 may have any suitable properties. For example, shaft 42 may be flexible and may have wires, tubes, or other features passing therethrough. Distal tip 44 of medical device 10, depicted in FIG. 1B, may be disposed at a distal end of shaft 42. As shown in FIG. 1B, distal tip 44 may include a distalmost face 46. Distalmost face 46 may define a working channel opening 48. The working channel may extend between port 24 and working channel opening 48, such that instruments or other devices may be passed through port 24, through the working channel, and out of working channel opening 48. An instrument extending distally of working channel opening 48 may be used to perform a medical procedure on a subject.

Distal tip 44 may also include imaging components, such as one or more lighting elements 50 and one or more imaging devices (e.g., camera) 52. Although two lighting elements 50 and one imaging device 52 are depicted in FIG. 1B, it will be appreciated that alternative numbers of lighting elements 50 and imaging device 52 may be utilized. Alternatively, lighting elements 50 and imaging device 52 may be combined into a single device. Lighting elements 50 may include LEDs or any suitable alternative light source. Imaging device 52 may be configured to take video and/or still images. Imaging device 52 may provide a signal to a display or monitor (not shown), so that an operator may view a visual image provided by imaging device 52 while navigating medical device 10 through a body lumen of a subject.

As depicted in FIG. 1B and described above, medical device 10 may be “forward-facing.” In other words, features of distal tip 44 (e.g., working channel opening 48, lighting elements 50, and imaging device 52) may face distally (i.e., forward of distalmost face 46. This disclosure also encompasses other configurations of distal tip 44. For example, medical device 10 may be “side-facing.” In a side-facing embodiment, working channel opening 48, lighting elements 50, and/or imaging device 52 may be disposed on a radially outer side of distal tip 44, so that they point in a radially outward direction, approximately perpendicularly to a longitudinal axis of insertion portion 14. Medical device 10 may additionally include some components that are forward-facing and other components that are side-facing.

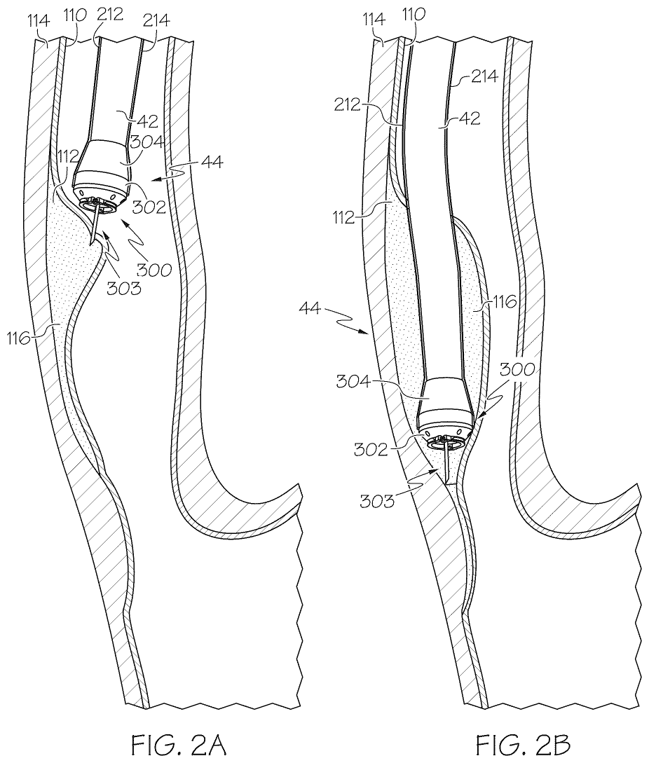

FIG. 2A depicts the shaft 42 of FIGS. 1A and 1B in a body lumen (e.g., an esophagus) of a subject. The distal tip 44 of the shaft 42 enters a second layer 112 (e.g., a submucosal space) between a third layer 114 (e.g., a muscularis) and a first layer 110 (e.g., a mucosal layer). To continue with the specific example tissues, the separation between the mucosal layer and the muscularis can be aided by a lifting agent 116. The lifting agent 116 can be injected into the submucosal space via, for example, one or more tools associated with the shaft 42.

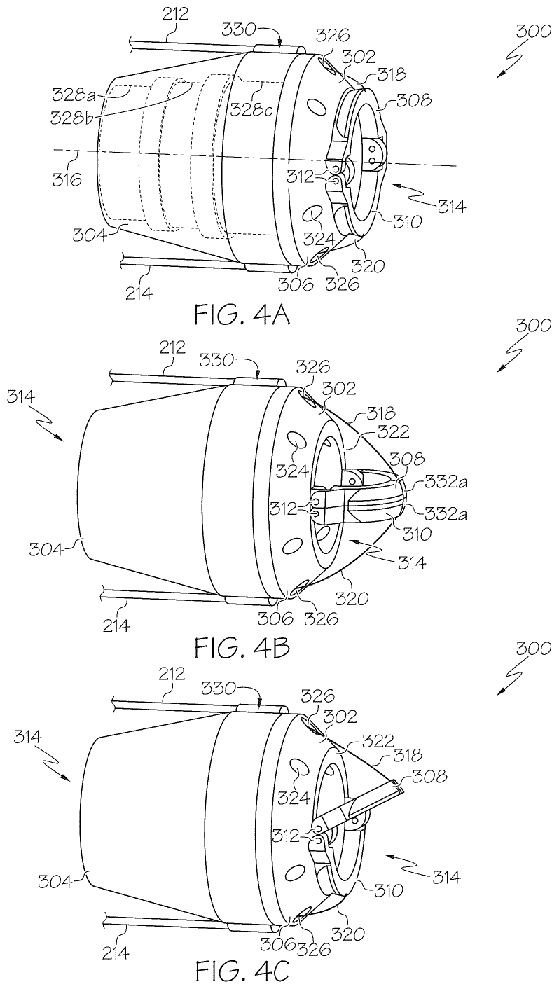

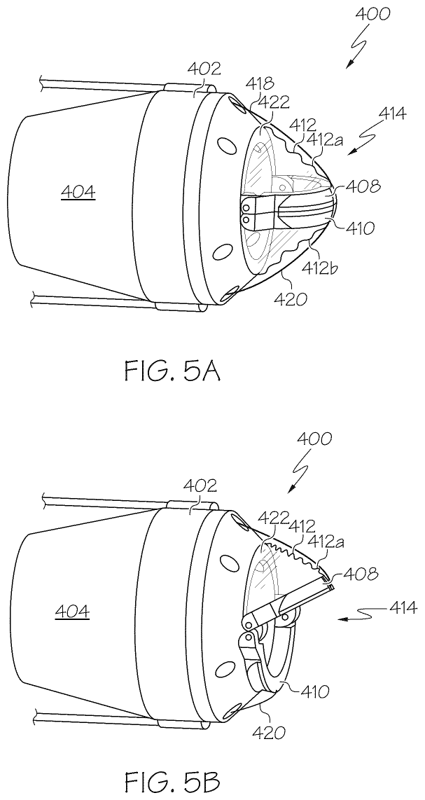

The distal tip 44 is capped or otherwise surrounded by a cap assembly 300 including a cap 302 and an adaptor 304. The adaptor 304 may be an endoscope adaptor, and the cap assembly 300 may cap or otherwise surround and/or extend distally beyond the distal tip 44 of the shaft 42. In these aspects, the cap assembly 300 may help to allow one or more features of the distal tip 44 and the shaft 42 to operate effectively within the subject while providing additional functionality, as explained in greater detail herein, especially with respect to FIGS. 4A-4C and 5A-5B. For example, one or more end effectors or implements 303 such as, for example, an electro-surgical knife may extend through the shaft 42 and pass through the cap assembly 300 for performing one or more actions in the body lumen of the subject.

One or more of the features of the cap assembly 300 of FIGS. 2A and 2B can be operable using a handle assembly 200, shown in FIG. 3. The handle assembly 200 can include a handle 202, a thumb ring 204, a first finger ring 206, and a second finger ring 208. The first finger ring 206 and the second finger ring 208 can be a first arm operator and a second arm operator for operating a first arm and a second arm of the cap assembly 300 of FIGS. 2A and 2B, as explained in greater detail herein.

Still referring to FIG. 3, a user may move the first finger ring 206 and the second finger ring 208 back and forth (e.g., proximally and distally) along the body of the handle 202 to move one or more moveable arms of the cap assembly 300. The first finger ring 206 and the second finger ring 208 can move along a portion of a length of the handle 202 within a slot 210, for example. The first finger ring 206 is operatively connected to a first actuation element (e.g., a wire, cable, thread, strand, etc. (not shown)). The second finger ring 208 is operatively connected to a second actuation element wire (e.g., a wire, cable, thread, strand, etc. (not shown)). The first actuation element may pass through a first actuation element tube 212, and the second actuation element may pass through a second actuation element tube 214. The first actuation element tube 212 and the second actuation element tube 214 may pass through the shaft 42 or may pass along the shaft 42 outside the shaft 42. The first actuation element tube 212 and the second actuation element tube 214 may operatively connect to the first finger ring 206 and the second finger ring 208 at a proximal end of the actuation elements. A distal end of each of the actuation elements may be operatively connected to arms on the cap assembly 300, for example, as shown in FIGS. 4A-4C and 5A-5B. Briefly referring to FIGS. 2A, 2B, and 3, the actuation element tubes 212, 214 can pass along an exterior of the shaft 42. For example, as discussed below with respect to FIGS. 4A-4C, actuation element tubes 212, 214 may extend along the exterior of the shaft 42 until the actuation element tubes 212, 214 reach an interface or portion of the cap assembly 300. Hence, a user can operate the handle assembly 200 to move components of the cap assembly 300 in conjunction with use of the medical device 10. The actuation element tubes 212, 214 can be made from a rigid, extruded material (e.g., plastic extrusion) or another material.

Still referring to FIG. 3, some embodiments of the handle assembly 200 may include a biasing device 216 (e.g., a spring). The biasing device 216 can bias the first finger ring 206 and/or the second finger ring 208 into a distal position, a proximal position, or another position to impart a biasing force to the first finger ring 206 and/or the second finger ring 208. As such, the biasing device 216 may impart a biasing force to the moveable features of the cap assembly 300 of FIGS. 2A and 2B.

FIGS. 4A-4C show the cap assembly 300 in greater detail. The cap assembly 300 includes the cap 302 and the adaptor 304. The cap 302 can be made from a plastic material (e.g., clear ABS plastic, polycarbonate, or a similar hard plastic material) or other rigid material. The cap 302 may include a body 306 and one or more arms, for example, a first arm 308 and a second arm 310. The arms 308, 310 may be hingeably or pivotally coupled to the body 306 at one or more hinges 312. In some embodiments, the arms 308, 310 can be coupled to the body 306 at one or more hinges and the hinges for arms 308, 310 may overlap or may be separate (e.g., slightly spaced apart). In embodiments, the cap 302 and the adaptor 304 may have a central lumen 314 along a central longitudinal axis 316 (only shown in FIG. 4A to improve clarity) of the cap assembly 300. The cap 302 may include one or more vent hole 324 and ports 326. Ports 326 may receive portions of a first wire 318 and a second wire 320. The wires 318, 320 may pass through respective ports 326 to the first wire tube 212 and the second wire tube 214, respectively. Additionally, the wires 318, 320 may be coupled to the arms 308, 310 at an apex 332a, 332b of each of the arms 308, 310. The apexes 332a, 332b may be the point on the arm 308, 310 that is furthest distal when each of the arms 308, 310 are in the closed position. With the wires 318, 320 being attached to the arms 308, 310 at the respective apexes 332a, 332b, movement of the wires 318, 320 may control of the movement and/or position of the arms 308, 310. The arms 308, 310 are rigid and can be made from a plastic, metal, or other rigid material.

The arms 308, 310 may move with respect to the central longitudinal axis 316 from a closed position to an open position. In the closed position, the arms 308, 310 may extend generally parallel to the central longitudinal axis 316. In the open position, the arms 308, 310 may extend generally perpendicular to the central longitudinal axis 316. The first arm 308 and the second arm 310 may be moved based on the motion of the first wire 318 and the second wire 320, for example, as controlled by a user using the first finger ring 206 and the second finger ring 208 of FIG. 3. Still referring to FIGS. 4A-4C, the wires 318, 320 may be made of a metal or other similarly stiff material and may operatively couple the arms 308, 310, respectively, with the first finger ring 206 (FIG. 3) and the second finger ring 208 (FIG. 3). The operative coupling between the finger rings 206, 208 and the arms 308, 310 helps to ensure that the arms 308, 310 move when a user moves the finger rings 206, 208. In these aspects, moving the finger ring(s) distally causes the respective arm(s) to move toward the closed position, and moving the finger ring(s) proximally causes the respective arm(s) to move toward the open position. Some embodiments of the cap assembly 300 have only one arm or more than two arms (e.g., three, four, five, etc. arms). In any of these aspects, the one or more arms can be controlled by one or more operators based on the principles of operation described herein.

Either or both of the first arm 308 and the second arm 310 can be a semi-circular arm, hingeably or pivotally connected to a distal surface 322 of the cap 302. In some embodiments, one or more surfaces of the arms 308, 310 may be sharpened, ridged, and/or otherwise textured, for example, to help enhance a tissue cutting and/or gripping effect of the arm, but in other embodiments the arms 308, 310 may not be sharpened, ridged, or otherwise textured.

As a user moves the wires using the handle assembly 200, the arms move back and forth between the closed and open positions to pull tissue apart and/or to perform blunt tissue dissection (e.g., through the submucosa during an endoscopic lumbar sympathectomy (ELS) procedure). With the arms in the fully open position, a user can use one or more end effectors or instruments (e.g., electrosurgical knife, balloon, injection solution, coag graspers, hemostasic clips, light(s), camera(s), etc.) inserted into the subject's body lumen with or as a part of, for example, the exemplary medical device 10 (FIGS. 1A-1B). The instruments can pass through one or more working channels and out of the working channel opening 48 the shaft 42 (FIGS. 1A, 2A, 2B) and through the central lumen 314 of the cap assembly 300. As such, a user may use one or more instruments with the cap assembly 300 installed on the shaft 42.

As alluded to above, the arms 308, 310 can be biased toward an open position or toward a closed position by biasing the finger rings 206, 208 in the handle assembly 200. For example, a spring or other biasing device may bias the finger rings 206, 208 toward a distal position or toward a proximal position. If the finger rings 206, 208 are biased toward the distal position, the arms 308, 310 would be biased toward the closed position. Conversely, if the finger rings 206, 208 are biased toward the proximal position, the arms 308, 310 would be biased toward the open position. In embodiments, both, one, or none of the arms 308, 310 may be biased. Similarly, in embodiments having more than two arms, all or fewer than all of the arms may be biased.

The vent holes 324 may be holes that may help permit the escape of gas or liquid through the vent holes 324, for example, during the operation of one or more tools at the distal end 44 of the shaft 42. For example, if smoke or other gas is produced during an endoscopic procedure, the smoke or other gas may pass through the vent holes 324 so that the smoke or other gas does not blur or otherwise obscure images captured by, for example, a camera (e.g., the camera 52) used in the medical device 10.

The adaptor 304 can be made of an elastic material (e.g., silicone) or another pliable or semi-rigid material. Additionally, the central lumen 314 of the adaptor 304 can have multiple radii 328a, 328b, 328c for adapting the cap assembly 300 to differently sized shafts 42. In some embodiments, the portions of the adaptor 304 near the most proximal radii may be flexible or the flexibility of the radii may increase in the distal direction with respect to the multiple radii 328a, 328b, 328c (with portions of the cap assembly 300 at radii 328a being most flexible). In these aspects, the adaptor 304 may be able to fit over shafts 42 of appropriate and/or differing sizes. The adaptor 304 may be press fit, snap fit, glued (or coupled with another adhesive), molded or otherwise joined to the cap 302 to form the cap assembly 300. In embodiments, the adaptor 304 and the cap 302 can be provided as a kit or a single assembly ready for installation on the distal end 44 of the shaft 42.

In embodiments, the adaptor 304 may have one or more channels 330 that are configured to interface with the ports 326 in the cap 302 and/or the wire tubes 212, 214 to provide a passage for the wires 318, 320. Thus, the wire tubes 212, 214 may generally extend from the channels 330 to the handle assembly 200, for example, along an exterior surface of the shaft 42. The adaptor 304 may have a tapering external profile, which may help to reduce interference between the adaptor 304 and the wire tubes 212, 214. In some embodiments, the cap 302 may taper from the proximal end to the distal surface 322 to help in delivery of the shaft 42 and with separation of tissue layers.

Referring to FIGS. 5A and 5B, another embodiment of a cap assembly 400 is shown. The cap assembly 400 includes a cap 402 and an adaptor 404. The cap 402 includes arms 408, 410, and arms 408, 410 may be hingeably or pivotally coupled to the cap 402. The cap 402 also includes a protective element or diaphragm 412 that extends between a distal surface 422 and the arms 408, 410. The diaphragm 412 may include two portions, for example, a first portion 412a that is connected between the first arm 408 and the distal surface 422, and a second portion 412b that is connected between the second arm 410 and the distal surface 422 of the cap 402. The diaphragm 412 may be moveable with the arms 408, 410. With the arms 408, 410 in a closed position (FIG. 5A), the diaphragm 412 may help to form a barrier between a central lumen 414 of the cap 402 and an external environment surrounding the cap 402. As such, with the arms 408, 410 in the closed position, the diaphragm 412 may help to prevent or inhibit tissue and/or other constituents of the external environment from entering the central lumen 414. In embodiments, the diaphragm 412 can have one or more pre-fabricated folds (e.g., concertina folds) that cause the diaphragm to extend and compress at the same folds each time the arms 408, 410 close and open, respectively. In the open position, the diaphragm 412 may be retracted so that instruments, cameras, and other tools/implements may be delivered through the shaft 42 and through the central lumen 414 without impairment or being affected by the diaphragm 412 or arms. As shown in FIGS. 5A and 5B, the cap 402 can include wires 418, 420 that couple the arms 408, 410 similarly to the arms 308, 310 of FIGS. 4A-4C.

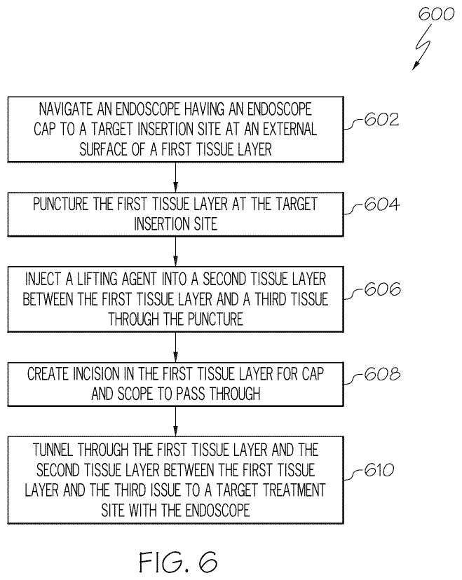

Referring now to FIG. 6, a method 600 of reaching a target treatment site using an endoscope including a dynamic or movable cap is shown. The method 600 could be performed at any treatment site within any layers of tissue of a subject and are discussed herein below with respect to an exemplary esophagus of a subject including, among other things, an epithelial layer, a mucosal layer, a submucosal layer, and a muscularis. At step 602, a user (e.g., a surgeon or other medical device operator) may navigate a shaft of an endoscope having a dynamic or movable endoscope cap, such as the cap 302 of FIGS. 4A-4C, to a target insertion site at an external surface of a first tissue layer (e.g., a mucosal layer or the epithelial layer). The endoscope may receive or include one or more tools or devices in its shaft that may aid with navigation (e.g., a camera, one or more illumination devices, a guide wire, etc.) The endoscope can be inserted into a body lumen (e.g., esophagus) through a naturally-occurring or surgically-created orifice. For example, a user may insert the endoscope through a mouth of the subject or an incision. Additionally, the endoscope may receive or include one or more tools or devices (e.g., one or more knives, for example, with an injection lumen) in its shaft that may treat the target insertion site

At step 604, with the endoscope at the target insertion site, the user may use one or more tools in the endoscope to puncture the first tissue layer at the target insertion site. For example, to use specific potential and non-limiting embodiments, the user may use an electrosurgical knife to puncture the mucosal layer. The puncture may pass through the mucosal layer to a submucosal layer. This puncture can enable access to a second tissue layer underlying the first tissue layer (e.g., the submucosal layer) with one or more tools of the endoscope.

At step 606, the user may inject a lifting agent into the second tissue layer between the first tissue layer and a third tissue layer (e.g., a muscularis). The user can, for instance, inject a lifting agent into the submucosal layer to cause the mucosal layer to separate from a muscularis. The lifting agent may be inserted via one or more tools inserted through a shaft of the endoscope. The lifting agent may be an inert substance that causes the first and/or second tissue layer (e.g., the mucosal layer) to separate from the third tissue layer (e.g., the muscularis), for example, to allow a distal portion of the endoscope to pass within the space between the first tissue layer and the third tissue layer.

At step 608, the user may use the endoscope cap to open the first tissue layer (e.g., the mucosal layer) at the target insertion site, for example, in order to gain access to the space between layers of tissue with the scope. For example, the user may make a small incision or other cut at the first tissue layer and then separate the first tissue layer from one or more of the second tissue layer and the third tissue layer. The arms of the cap 302 can be used to increase a size of the incision, for example, to help enlarge the size of the incision such that the cap 302 and the scope 42 can pass through the incision.

At step 610, the user may tunnel between the first tissue layer and the second tissue layer. The tunneling can include using an endoscope with a dynamic endoscope cap such as the cap 302 of FIGS. 4A and 4B. The dynamic endoscope cap can include a body with a central lumen about a central longitudinal axis, a first arm, and a second arm. The first arm may be hingeably or pivotally coupled to the body and operably coupled to the first arm operator by a first wire, and the second arm may be hingeably or pivotally coupled to the body and operably coupled to the second arm operator by a second wire.

During the tunneling, the arms of the cap may be moved using a handle assembly such as the handle assembly 200 of FIG. 3. In embodiments, a user may manipulate the handle assembly to cause one or more of the arms to move about the respective pivot axis, thereby displacing the subject's tissue from in front of (e.g., distal to) the endoscope and moving the tissue to a side of the endoscope to tunnel through the tissue (e.g., separating the layers of tissue). As the tissue is displaced, a user can move the endoscope through (e.g., distally through) the portion of the subject's anatomy further toward the target treatment site until the user reaches the target treatment site with the endoscope. At the target treatment site, the user can use one or more tools (e.g., knife, camera, etc.) to treat the target treatment site. For example, a user may perform a myotomy in an esophagus of the subject by extending a knife or other instrument through the endoscope once at the treatment site.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
a handle comprising:
a first arm operator; and
a second arm operator; and
a cap comprising:
a body having a central lumen about a central longitudinal axis;
a first arm hingeably coupled to the body and operably coupled to the first arm operator by a first wire; and
a second arm hingeably coupled to the body and operably coupled to the second arm operator by a second wire; wherein
the first arm operator is moveable in the handle to move the first arm between a closed position with the first arm generally parallel to the central longitudinal axis and an open position with the first arm generally perpendicular to the central longitudinal axis,
the second arm operator is moveable in the handle to move the second arm between a closed position with the second arm generally parallel to the central longitudinal axis and an open position with the second arm generally perpendicular to the central longitudinal axis.

2. The medical device of claim 1, wherein the first arm and the second arm are semi-circular arms.

3. The medical device of claim 2, wherein the first wire is connected to the first semi-circular arm at an apex of the first semi-circular arm, and wherein the second wire is connected to the second semi-circular arm at an apex of the second semi-circular arm.

4. The medical device of claim 2, wherein the first arm and the second arm comprise metal or a hard plastic material.

5. The medical device of claim 1, wherein the first arm and the second arm are biased toward an open position.

6. The medical device of claim 5, wherein the first arm is biased toward an open position by a first spring in the handle that biases the first arm operator, and wherein the second arm is biased toward an open position by a second spring in the handle that biases the second arm operator.

7. The medical device of claim 1, wherein the cap comprises a proximal portion configured to overlap with a distal portion of an insertion device.

8. The medical device of claim 7, further comprising a distal portion configured to extend distally of the distal portion of the insertion device.

9. The medical device of claim 7, wherein the insertion device is an endoscope, and wherein the cap comprises:
an endoscope adaptor; and
an endoscope cap for capping a shaft of the endoscope.

10. The medical device of claim 9, wherein the endoscope adaptor is made of an elastic material, and wherein the endoscope cap is made of a plastic material.

11. The medical device of claim 10, wherein the endoscope adaptor is made of silicone, and wherein the endoscope cap is made of a clear material including ABS plastic, polycarbonate, or a similar plastic.

12. The medical device of claim 1, wherein the first wire is surrounded by a first tube, wherein the second wire is surrounded by a second tube, and wherein proximal ends of the first tube and the second tube are connected to the handle.

13. The medical device of claim 12, wherein the first tube and the second tube are each made from a plastic extrusion.

14. The medical device of claim 1, further comprising:
a diaphragm that is connected between the first arm and the second arm and an endoscope cap, wherein the diaphragm moveable with the first arm and the second arm to form a barrier between the central lumen of the cap and an external environment with the first arm and the second arm in the closed position.

15. The medical device of claim 1, wherein the cap includes one or more vent holes through the body of the cap.

16. A cap for an insertion device comprising:
a body having a central lumen about a central longitudinal axis;
a first arm hingeably coupled to the body and operably coupled to a first wire; and
a second arm hingeably coupled to the body and operably coupled to a second wire; wherein
the first arm is moveable between an open position with the first arm generally perpendicular to the central longitudinal axis and a closed position with the first arm generally parallel to the central longitudinal axis, and
the second arm is moveable between an open position with the second arm generally perpendicular to the central longitudinal axis and a closed position with the second arm generally parallel to the central longitudinal axis.

17. The cap of claim 16, wherein
the first arm is operably coupled to a first arm operator in a handle by a first actuation element;
the second arm is operably coupled to a second arm operator in the handle by a second actuation element;
the first arm operator is moveable in the handle to move the first arm; and
the second arm operator is moveable in the handle to move the second arm.

18. The cap of claim 16, further comprising a diaphragm that is connected between the first arm and the second arm and an endoscope cap, wherein the diaphragm is moveable with the first arm and the second arm to form a barrier between the central lumen of the cap and an external environment with the first arm and the second arm in the closed position.

19. A method of reaching a target treatment site using an endoscope comprising:
navigating an endoscope having an endoscope cap to a target insertion site at an external surface of a first tissue layer;
puncturing the first tissue layer at the target insertion site;
injecting a lifting agent between the first tissue layer and a third tissue layer through the puncture;
tunneling through the first tissue layer and the second tissue layer between the first tissue layer and the third tissue layer to a target treatment site with the endoscope, wherein
the tunneling includes tunneling using the endoscope cap including a body having a central lumen about a central longitudinal axis, a first arm hingeably coupled to the body and operably coupled to the first arm operator by a first wire, and a second arm hingeably coupled to the body and operably coupled to the second arm operator by a second wire.

20. The method of claim 19, wherein transitioning the first arm and the second arm from a closed configuration to an open configuration includes an operator urging the first arm operator and the second arm operator proximally on a handle to (1) move the first arm with the first arm operator and (2) move the second arm with the second arm operator to tunnel between the first tissue layer and the third tissue layer.
